(19) United States
(12) Patent Application Publication
(10) Pub. No.: US 2009/0143340 A1
(43) Pub. Date: Jun. 4, 2009

(54) Pyrocatechin Derivatives

(76) Inventors: Gottfried Sedelmeier, Schallstadt (DE); Kurt Nebel, Hochwald (CH); Siem Jacob Veenstra, Lorrach (DE); Janos Zergenyi, Seltisberg (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/994,919

(22) PCT Filed: Jul. 10, 2006
(86) PCT No.: PCT/EP2006/006731

§ 371 Date: Jan. 7, 2008

(30) Foreign Application Priority Data
Jul. 11, 2005 (EP) 05014950.9

(51) Int. Cl. A61K 31/655; A61K 31/365; A61K 31/12; C07C 69/96; C07C 49/825

(52) U.S. Cl. 514/151; 514/473; 514/512; 514/689; 549/305; 552/8; 558/268; 568/337

(57) ABSTRACT

Pyrocatechin derivatives of formula I

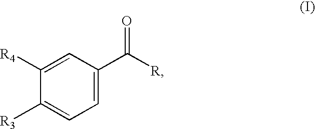

wherein R illustrates a group of formulae Ia, Ib, Ic or Id

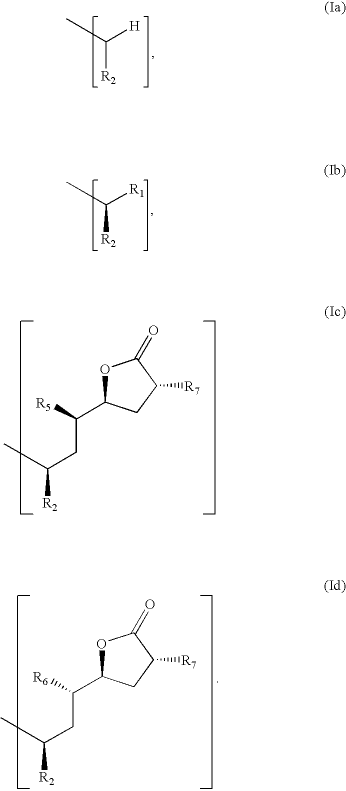

R₁ is 4-halogen-but-2-enyl, R₂ is lower alkyl or cycloalkyl, R₃ is lower alkoxy and R₄ is lower alkoxy lower alkoxy, or, where R is a group of formula (Ia), it is hydroxy, hydroxy lower alkoxy or a group of formula Ie

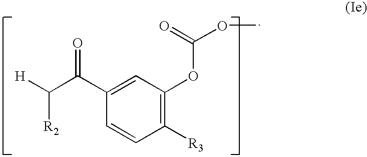

R₅ is reactive esterified hydroxy, R₆ is azido and R₇ is lower alkyl, lower alkenyl, cycloalkyl or aryl lower alkyl, and their salts, are valuable intermediates in the production of active ingredients for medicaments.

The invention relates to new compounds of formula I

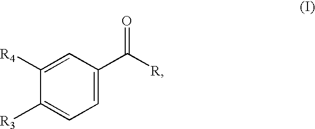

wherein

R illustrates a group of formulae Ia, Ib, Ic or Id

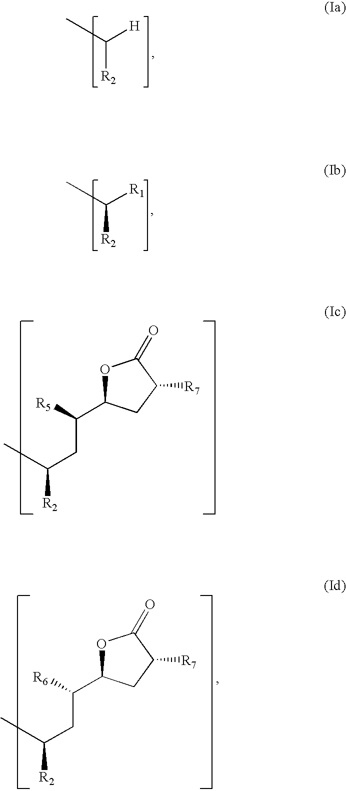

R₁ is 4-halogenbut-2-enyl,

R₂ signifies lower alkyl or cycloalkyl,

R₃ is lower alkoxy,

R₄ illustrates lower alkoxy lower alkoxy, or where R is a group of formula (Ia), it is hydroxy, hydroxy lower alkoxy or a group of formula Ie

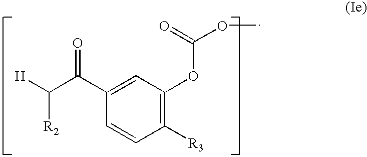

R₅ signifies reactive, esterified hydroxy,

R₆ is azido and

R₇ illustrates lower alkyl, lower alkenyl, cycloalkyl or aryl lower alkyl, and their salts, a process for their preparation and their use as intermediates in the production of active ingredients for medicaments.

Halobut-2-enyl is, for example, 4-bromobut-2-enyl or 4-chlorobut-2-enyl, especially 4-bromobut-2-enyl.

Aryl lower alkyl is, for example, phenyl lower alkyl which is either unsubstituted or substituted in the phenyl moiety by lower alkyl, lower alkoxy, halogen and/or nitro.

Cycloalkyl has, for example, 3 to 8, preferably 3 ring members, and signifies primarily cyclopropyl, or also cyclobutyl, cyclopentyl or cyclohexyl.

Reactive esterified hydroxy is, for example, halogen or sulfonyloxy, such as lower alkane-sulfonyloxy, halogen lower alkanesulfonyl, lower alkanesulfonyloxy; or benzenesulfonyloxy or naphthalenesulfonyloxy either unsubstituted or substituted by lower alkyl, halogen and/or nitro.

Hereinbefore and hereinafter, by lower radicals and compounds is understood, for example, those radicals and compounds having up to and including 7, preferably up to and including 4 carbon atoms (C-atoms).

Halogen is, for example, halogen with an atomic number from 19 to 35, such as chlorine or in particular bromine.

Halogen lower alkanesulfonyloxy is, for example, polyhalogen-C₁-C₄-alkanesulfonyloxy, such as trifluoromethanesulfonyloxy.

Lower alkanesulfonyloxy is, for example C₁-C₄-alkanesulfonyloxy, such as methanesulfonyloxy, ethanesulfonyloxy, propanesulfonyloxy or butanesulfonyloxy.

Lower alkenyl may be straight-chained or branched and/or bridged, and is for example a corresponding C₂-C₄-alkenyl, such as allyl.

Lower alkenesulfonyloxy is, for example C₂-C₄-alkenesulfonyloxy, such as ethenesulfonyloxy.

Lower alkyl may be straight-chained or branched and/or bridged and is, for example, corresponding C₁-C₇-alkyl, especially C₁-C₄-alkyl, such as methyl, ethyl, propyl, isopropyl, butyl, isobutyl, secondary butyl, tertiary butyl or a pentyl, hexyl or heptyl group. Lower alkyl R₂ or R₅ is, for example, methyl or in particular branched C₃-C₇-alkyl, such as propyl.

Lower alkoxy is, for example, C₁-C₇-alkoxy, preferably C₁-C₄-alkoxy, such as methoxy, ethoxy, propyloxy, isopropyloxy, butyloxy, isobutyloxy, secondary butyloxy, tertiary butyloxy, pentyloxy or a hexyloxy or heptyloxy group.

Hydroxy lower alkoxy is, for example, hydroxy-C₂-C₄-alkoxy, such as 2-hydroxyethoxy, 3-hydroxypropyloxy or 4-hydroxybutyloxy, especially 3-hydroxypropyloxy or 4-hydroxybutyloxy.

Lower alkoxy lower alkoxy is, for example, C₁-C₄-alkoxy-C₂-C₄-alkoxy, such as 2-methoxy-, 2-ethoxy- or 2-propyloxyethoxy, 3-methoxy- or 3-ethoxypropyloxy or 4-methoxybutyloxy, especially 3-methoxypropyloxy or 4-methoxybutyloxy.

Phenyl lower alkyl is, for example, phenyl-C₁-C₄-alkyl, preferably 1-phenyl-C₁-C₄-alkyl, such as benzyl or 1-phenylethyl.

Salts are especially phenolate salts of compounds of formula I, wherein R₄ is hydroxy with appropriate bases, such as metal salts derived from metals of group Ia, Ib, IIa and IIb of the periodic table of elements, e.g. alkali metal salts, especially lithium, sodium or potassium salts, alkaline earth metal salts, for example magnesium or calcium salts, also zinc salts.

The invention was based on the problem of developing an improved method of obtaining compounds of formula II

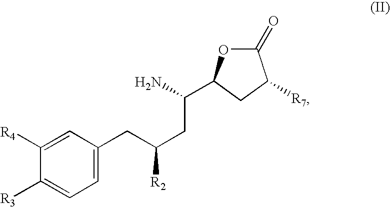

wherein R₂, R₃ and R₇ have the indicated significances, and R₄ signifies lower alkoxy lower alkoxy, which are valuable intermediates for the production of active ingredients for medicaments. For example, compounds of formula II according to EP-678503 are used as preferred intermediates for the production of compounds of formula

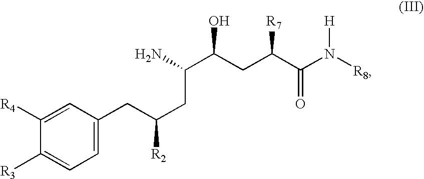

wherein R₂, R₃, R₄ and R₇ have the indicated significances, R₄ signifies lower alkoxy lower alkoxy, and R₈ signifies lower alkyl, cycloalkyl, optionally aliphatically esterified or etherified hydroxy lower alkyl, optionally N-lower alkanoylated, N-mono- or N,N-dilower alkylated amino lower alkyl, or amino lower alkyl N,N-disubstituted by lower alkylene, hydroxy-, lower alkoxy- or lower alkanoyloxy lower alkylene, optionally N′-lower alkanoylated or N′-lower alkylated aza lower alkylene, oxa lower alkylene or optionally S-oxidised thia lower alkylene; optionally esterified or amidated carboxy lower alkyl, optionally esterified or amidated dicarboxy lower alkyl, optionally esterified or amidated carboxy(hydroxy) lower alkyl, ooptionally esterified or amidated carboxycycloalkyl lower alkyl, cyano lower alkyl, lower alkanesulfonyl lower alkyl, optionally N-mono- or N,N-di-lower alkylated thiacarbamoyl lower alkyl, optionally N-mono- or N,N-di-lower alkylated sulfamoyl lower alkyl; or a heteroaryl radical which is bonded by a C-atom and is optionally hydrated and/or oxo-substituted; or a lower alkyl which is substituted by a heteroaryl radical which is bonded by a C-atom and is optionally hydrated and/or oxo-substituted, and their salts.

The intermediates of formula II are at present prepared in a four-step synthesis from 2-lower alkoxyphenols on the one hand and phenyl alaninol on the other hand, in accordance with the following scheme.

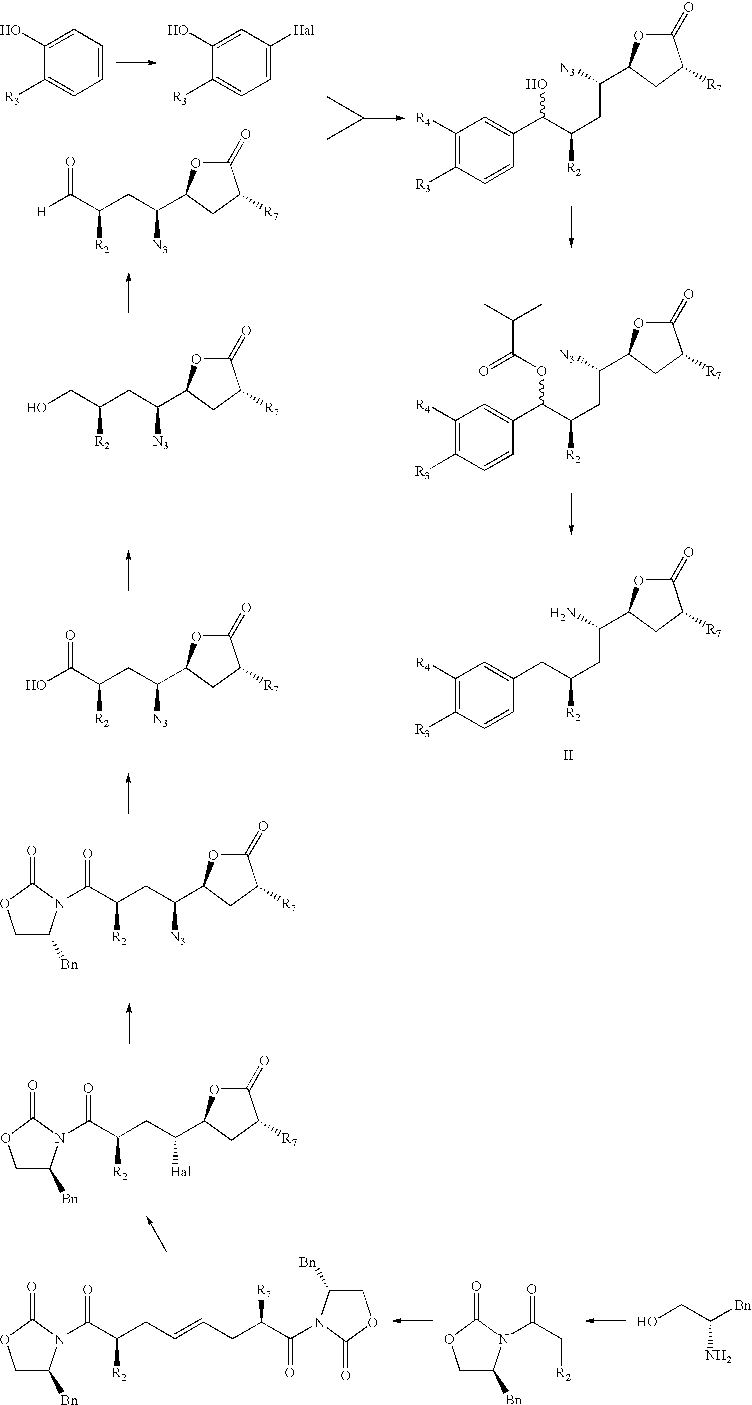

On a technical scale, the production of these starting materials and their further processing to the intermediates II is extremely complex, technical and, from a safety point of view, difficult to control, and expensive. There was therefore an urgent need for an improved and technically practicable alternative process for the production of compounds of formula II.

The solution to the problem according to the invention is based on the consideration that instead of halogenated, it is more advantageous to use acylated 2-lower alkoxyphenols, which already contain part of the side chain, and is based on the surprising discovery that the acylation of 2,2′-dilower alkoxy diphenylcarbonates takes place with high selectivity in p-position to the lower alkoxy groups. The process according to the invention uses methods which can be carried out more easily on a technical scale and are less problematic from an ecological and safety point of view than the known process.

The solution, according to the invention, to the problem of producing the intermediates II is clarified by the following reaction scheme.

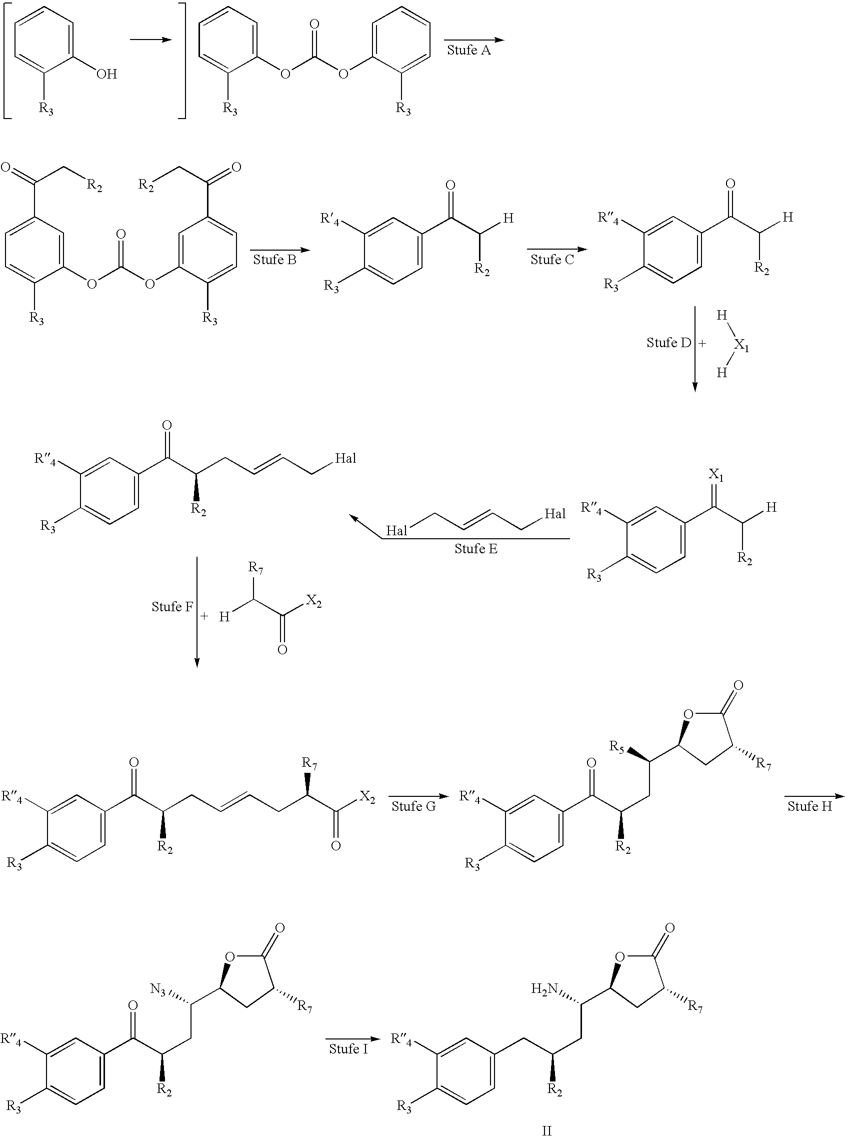

In a variation of this reaction sequence, steps E and F can also be exchanged by reacting the 1,4-dihalogen-but-2-ene in accordance with the reaction scheme

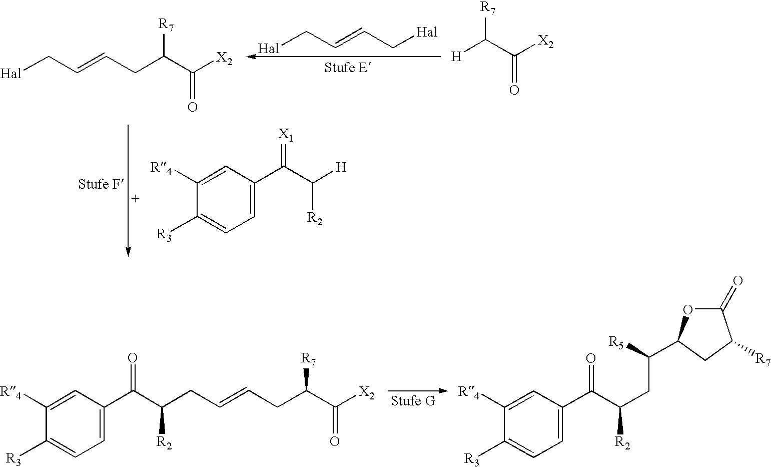

first of all with the chiral amide and then with the chiral ketimine which is obtainable according to steps D+E.

In the depicted reaction schemes, R′₄ signifies hydroxy lower alkoxy, R″₄ signifies lower alkoxy lower alkoxy, X₁ signifies the imino group derived from a chiral primary amine which is customary for the purpose of stereoselective synthesis, such as (S)-1-phenylethylamine or (S)-2-amino-1-methoxy-3-phenylpropane, and X₂ signifies a chiral amino, amido or urethane group which is customary for the purpose of stereoselective synthesis, or a chiral alcohol group. Compounds containing groups of this kind are, for example, pseudoephedrine, 4(S)-benzyl-2-oxo-oxazolidine, camphor sultam, borneol, intermediately-protected (S,S)- or (R,R)-2-aminophenylpropanediol, menthol, 8-phenylmenthol, pantolactone and the like.

In addition to the compounds of formula I prepared according to the invention, the invention also relates to steps A, B, C, D+E, F+G or F′+F′+G and H for their production, and the whole process, comprising these steps, for producing compounds of formula I, wherein R is a group of formulae Ic and Id, R₂ is lower alkyl or cycloalkyl, R₃ is lower alkoxy, R₄ is lower alkoxy lower alkoxy, R₅ is reactive esterified hydroxy, especially halogen, R₆ is azido and R₇ is lower alkyl, lower alkenyl, cycloalkyl or aryl lower alkyl.

The use of compounds of formula I for the production of intermediates of formula II, in order to produce active ingredients for medicaments, of formula III, described in EP-678503, is a further object of the present invention.

The invention relates primarily to compounds of formula I, wherein

R illustrates a group of formulae Ia, Ib, Ic or Id,

R₁ is 4-halogenbut-2-enyl,

R₂ is lower alkyl or 3- to 8-membered cycloalkyl,

R₃ is lower alkoxy,

R₄ illustrates lower alkoxy lower alkoxy, or where R is a group of formula (Ia), it is hydroxy, hydroxy lower alkoxy or a group of formula Ie.

R₅ is halogen or sulfonyloxy, such as lower alkane sulfonyloxy, halogen lower alkane sulfonyl, lower alkane sulfonyloxy; or benzene sulfonyloxy or naphthalene sulfonyloxy which is unsubstituted or substituted by lower alkyl, halogen and/or nitro.

R₆ is azido and

R₇ is lower alkyl, lower alkenyl, 3- to 8-membered cycloalkyl, or phenyl lower alkyl which is unsubstituted or substituted in the phenyl moiety by lower alkyl, lower alkoxy, halogen and/or nitro,

and their salts, a process for their preparation and their use as intermediates in the production of active ingredients for medicaments.

The invention preferably relates to compounds of formula I, wherein

R illustrates a group of formulae Ia, Ib, Ic or Id,

R₁ is 4-halogenbut-2-enyl,

R₂ is branched C₃-C₇-alkyl, such as propyl,

R₃ is C₁-C₄-alkoxy, such as methoxy,

R₄ is C₁-C₄-alkoxy-C₂-C₄-alkoxy, such as 3-methoxypropyloxy, or where R is a group of formula (Ia), it is hydroxy, hydroxy-C₂-C₄-alkoxy, such as 3-hydroxypropyloxy, or a group of formula Ie,

R₅ is halogen of atomic numbers up to and including 35, such as bromine,

R₆ is azido or amino and

R₇ is branched C₃-C₇-alkyl, such as propyl,

and their salts, a process for their preparation and their use as intermediates in the production of active ingredients for medicaments.

The invention relates specifically to the compounds of formula I and their salts named in the examples, processes for their preparation, and their use as intermediates for producing active ingredients for medicaments.

The process according to the invention for the preparation of the compounds of formula I is characterized in that

a) in order to produce compounds of formula I, wherein R is a group of formula Ia, R₂ is lower alkyl or cycloalkyl, R₃ is lower alkoxy and R₄ is a group of formula Ie,

a compound of formula IV

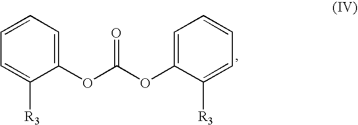

wherein R₃ has the above significance, is condensed with a compound of formula V

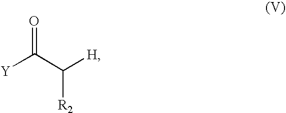

wherein R₂ has the above significance and Y is a reactive esterified hydroxy group,

b) in order to produce a compound of formula I, wherein R is a group of formula Ia, R₂ is lower alkyl or cycloalkyl, R₃ is lower alkoxy and R₄ is hydroxy, a compound of formula VI,

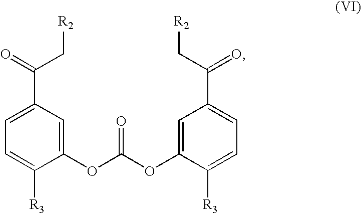

wherein R₂ and R₃ have the above significances, undergoes solvolysis to form the corresponding compounds of formula I, wherein R₄ is hydroxy,

c) in order to produce a compound of formula I, wherein R is a group of formula Ia, R₂ is lower alkyl or cycloalkyl, R₃ is lower alkoxy and R₄ is hydroxy lower alkoxy R′₄, in a compound of formula VII,

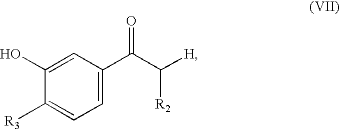

wherein R₂ and R₃ have the above significances, the hydroxy group is converted into hydroxy lower alkoxy R′₄,

d) in order to produce a compound of formula I, wherein R is a group of formula Ia, R₂ is lower alkyl or cycloalkyl, R₃ is lower alkoxy and R₄ is lower alkoxy lower alkoxy R″₄, in a compound of formula VIII,

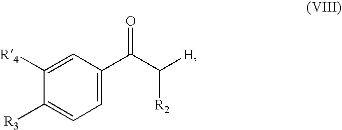

wherein R₂ and R₃ have the above significances and R′₄ is hydroxy lower alkoxy, the hydroxy lower alkoxy group is converted into lower alkoxy lower alkoxy R″₄,

e) in order to produce compounds of formula I, wherein R is a group of formula Ib, R₁ is 4-halogen-but-2-enyl, R₂ is lower alkyl or cycloalkyl, R₃ is lower alkoxy and R₄ is lower alkoxy lower alkoxy R″₄, a compound of formula IX

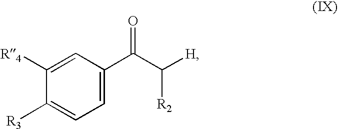

wherein R₂, R₃ and R″₄ have the above significances, is reacted first of all with a chiral primary amine which is customary for the purpose of stereoselective synthesis, and then with a 1,4-dihalogen-but-2-ene,

f) in order to produce compounds of formula I, wherein R is a group of formula Ic, R₂ is lower alkyl or cycloalkyl, R₃ is lower alkoxy, R₄ is lower alkoxy lower alkoxy R″₄, R₅ is reactive esterified hydroxy and R₇ is lower alkyl, lower alkenyl, cycloalkyl or aryl lower alkyl,

a compound of formula X,

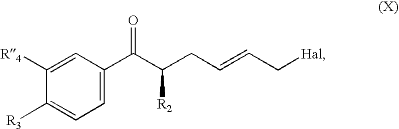

wherein R₂, R₃ and R″₄ have the above significances, and Hal is halogen, is reacted with a compound of formula XI

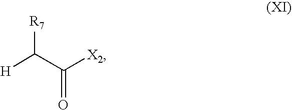

in which R₇ has the indicated significance and X₂ is a chiral amino, amido or urethane group which is customary for the purpose of stereoselective synthesis, or is a chiral alcohol group; or a 1,4-dihalogen-but-2-ene is reacted firstly with a compound of formula XI and then with the reaction product from the reaction of a compound of formula IX

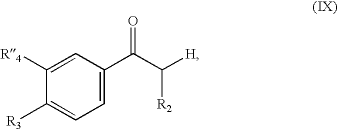

with a chiral primary amine of formula XII, which is customary for the purpose of stereoselective synthesis,

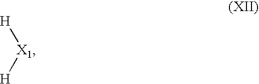

and the reaction product of formula XIII

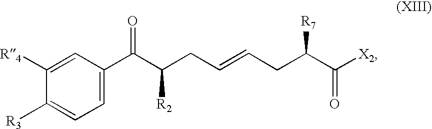

is condensed intramolecularly with lactonisation to form the corresponding compounds of formula I, wherein R is a group of formula Ic and R₅ is halogen, and if desired, halogen R₅ is converted stereoselectively into another reactive etherified hydroxy group R₅, and/or

g) in order to produce compounds of formula I, wherein R is a group of formula Id, R₂ is lower alkyl or cycloalkyl, R₃ is lower alkoxy, R₄ is lower alkoxy lower alkoxy R″₄, R₆ is azido and R₇ is lower alkyl, lower alkenyl, cycloalkyl or aryl lower alkyl, in a compound of formula XIV

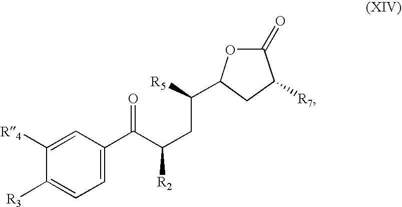

wherein R₂, R₃, R″₄ and R₇ have the above significances, and R₅ is reactive esterified hydroxy, reactive esterified hydroxy R₅ is replaced stereospecifically by azido, and if desired, a free compound obtainable according to the method is converted into a salt, or a salt obtainable according to the method is converted into the free compound or into another salt.

In starting substances of formula according to process variant a), reactive esterified hydroxy signifies, for example, hydroxy which is esterified with a hydrohalic acid or an organic sulfonic acid, such as halogen, especially chlorine, or optionally halogenated lower alkane sulfonyloxy, such as methane sulfonyloxy or trifluoromethane sulfonyloxy.

The reaction of compounds of formulae IV and V is effected in the usual manner, preferably in the presence of an acidic condensation agent, for example a Lewis acid, especially aluminium trichloride, advantageously in an inert solvent, such as a halogenated hydrocarbon, such as methylene chloride, if necessary whilst cooling, preferably in a temperature range from ca. 5° C. to ca. 30° C., for example at room temperature.

The solvolysis of compounds of formula VI according to process variant b) is effected by conventional solvolysis processes, preferably under the conditions of a basic hydrolysis, for example by treatment with sodium hydroxide in an aqueous lower alkanol, such as methanol/water mixtures, advantageously with heating, preferably in a temperature range from ca. 55° C. to ca. 90° C., for example at boiling temperature.

The conversion of the hydroxy group R₄ into hydroxy lower alkoxy according to process variant c) may take place in conventional manner, for example by a reaction with a reactive monoester, such as a hydrohalic acid ester or sulfonic acid ester of a lower alkanediol, such as a ω-hydroxy lower alkyl halide, if necessary in the presence of a basic condensation agent, such as an alkali metal or alkaline earth metal hydroxide or an alkali metal carbonate, advantageously in a solvent which is inert towards the reaction components, if necessary with heating, for example in a temperature range of ca. 60° C. to ca. 100° C., preferably by reacting with a ω-hydroxy lower alkyl halide in the presence of potassium carbonate in boiling acetonitrile.

Conversion of the hydroxy lower alkoxy group R′₄ into lower alkoxy lower alkoxy R″₄ according to process variant d) may take place in conventional manner, for example by a reaction with a reactive ester of a lower alkanol, such as corresponding halides, sulfates or optionally halogenated lower alkane sulfonates, such as methane sulfonates or trifluoro-methane sulfonates, if necessary in the presence of a basic condensation agent, such as an alkali metal or alkaline earth metal hydroxide, advantageously in a solvent which is inert towards the reaction components, if necessary with heating, for example in a temperature range of ca. 30° C. to ca. 60° C., preferably by reacting with a di-lower alkyl sulfate in the presence of potassium hydroxide in toluene at ca. 40° C.

Suitable amines for the reaction of compounds of formula IX according to process variant e) are chiral primary amines which are customary for the purpose of stereoselective synthesis, such as (S)-1-phenylethylamine or (S)-2-amino-1-methoxy-3-phenylpropane. The reaction of compounds of formula IX firstly with a chiral primary amine and then with a 1,4-dihalobut-2-ene is preferably carried out in a one-pot reaction without isolation of the intermediates in a solvent which is suitable for both steps, such as a solvent forming an azeotropic mixture with water, e.g. benzene or preferably toluene, if necessary adding one further or several further solvent(s), and in the second step adding a basic condensation agent, such as an alkali metal amide derivative, such as a N,N-bis(tri-lower-alkylsilyl)-alkali metal amide, as well as a tertiary amide or lactam. Preferably, a solution of the compound of formula IX and the chiral amine is heated in toluene, firstly whilst distilling the reaction water, e.g. in a water separator, then concentrating to ca. two thirds to one half, then the basic condensation agent is added, for example a solution of a N,N-bis(tri-lower-alkylsilyl)-alkali metal amide, preferably N,N-bis(trimethylsilyl)-lithium amide, in an ether-like solvent, such as tetrahydrofuran, and if necessary a tertiary amide or lactam is added, e.g. dimethylpropylene urea, then a solution of 1,4-dihalobut-2-ene is added whilst cooling, for example in a temperature range of ca. −10° C. to ca. +10° C., and heated to room temperature, and working up is effected under slightly acidic conditions.

In the reaction with compounds of formula X or 1,4-dihalobut-2-enes according to process variant f), suitable compounds of formula XI are, for example, those in which X₂ signifies a chiral amino, amido, urethane or alcohol group. Compounds containing groups of this kind are, for example, chiral amines, amides, urethanes and alcohols, which are customary for the purpose of stereoselective synthesis, for example pseudoephedrine, 4(S)-benzyl-2-oxo-oxazolidine, camphor sultam, borneol, intermediately-protected (S,S)- or (R,R)-2-aminophenylpropanediol, menthol, 8-phenylmenthol, pantolactone and the like.

The reaction of compounds of formulae X and XI, likewise the reaction of 1,4-dihalo-but-2-enes and compounds of formula XI, preferably takes place in the presence of a basic condensation agent, for example an alkali metal amide derivative, such as a N,N-bis-(tri-lower-alkylsilyl)-alkali metal amide, e.g. N,N-bis-(trimethylsilyl)-lithium amide, as well as a tertiary amide or lactam, advantageously in a solvent which is inert towards the reaction components, such as toluene, if necessary whilst cooling, e.g. in a temperature range from ca. −10° C. to ca. +10° C., in an ether-like solvent, such as tetrahydrofuran, adding a solution of the 1,4-dihalo-but-2-ene whilst cooling, for example in a temperature range from ca. −10° C. to ca. +10° C. and working up under slightly acidic conditions, and heating to room temperature and working up under slightly acidic conditions.

Suitable amines for the reaction of compounds of formula IX are, for example, chiral primary amines which are customary for the purpose of stereoselective synthesis, such as (S)-1-phenylethylamine or (S)-2-amino-1-methoxy-3-phenylpropane.

The reaction of compounds of formula IX firstly with a chiral primary amine and then with an intermediate formed by reacting a 1,4-dihalobut-2-ene with a compound of formula XI, is preferably carried out in a one-pot reaction without isolation of the intermediates in a solvent which is suitable for both steps, such as a solvent forming an azeotropic mixture with water, e.g. benzene or preferably toluene, if necessary adding one further or several further solvent(s), and in the second step adding a basic condensation agent, such as an alkali metal amide derivative, such as a N,N-bis(tri-lower-alkylsilyl)-alkali metal amide, as well as a tertiary amide or lactam. Preferably, a solution of the compound of formula IX and the chiral amine of formula XII is heated in toluene, firstly whilst distilling the reaction water, e.g. in a water separator, then concentrating to ca. two thirds to one half, then the basic condensation agent is added, for example a solution of a N,N-bis(tri-lower-alkylsilyl)-alkali metal amide, preferably N,N-bis(trimethylsilyl)-lithium amide, in an ether-like solvent, such as tetrahydrofuran, and if necessary a tertiary amide or lactam is added, e.g. dimethylpropylene urea, then a solution of the intermediate formed by reacting a 1,4-dihalobut-2-ene with a compound of formula XI is added whilst cooling, for example in a temperature range of ca. −10° to ca. +10° C., heated to room temperature, and working up is effected under slightly acidic conditions.

The intramolecular condensation of compounds of formula XIII with lactonisation is carried out, for example, by treatment with a halogenation agent, such as a N-halo-dicarboxylic acid imide, for example N-bromosuccinimide, advantageously in a two-phase solvent system, such as dichloromethane/water. The stereoselective exchange of halogen R₅ into another reactive etherified hydroxy group R₅ may be carried out in a conventional manner.

The stereoselective replacement of halogen R₅ with azido according to process variant g) is effected in the usual manner, for example by a reaction with a metal or ammonium azide, for example with sodium azide, preferably in the presence of a quaternary nitrogen base, such as a quaternary aliphatic amine, e.g. N,N,N-tricapryl-N-methyl-ammonium chloride, if necessary whilst heating, e.g. in a temperature range from ca. 50° C. to ca. 100° C., advantageously in a two-phase solvent system, such as toluene/water.

According to the intended use of compounds of formula I prepared according to the invention, a further object of the invention is a process for the production of compounds of formula II

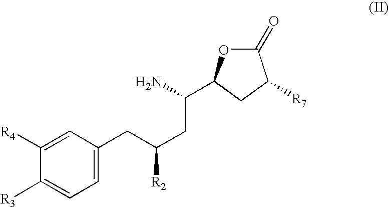

wherein R₂, R₃, R₄ and R₇ have the indicated significances, and their salts. This is characterized in that

h) a compound of formula I, wherein R is a group of formula Id, R₂ is lower alkyl or cycloalkyl, R₃ is lower alkoxy, R₄ is lower alkoxy lower alkoxy, R₆ is azido and R₇ is lower alkyl, lower alkenyl, cycloalkyl or aryl lower alkyl, the azido group R₆ is reduced to amino, and at the same time or subsequently, the benzoyl group is reduced to the corresponding benzyl group, for example by catalytic hydrogenation, for example in the presence of a palladium catalyst, such as palladium on carbon, at normal pressure and at room temperature, advantageously in a lower alkanol, such as ethanol, especially in a mixture with an aliphatic amino-alkanol, such as ethanolamine.

Salts of compounds of formula I and II, which are obtainable by the process, can be converted in known manner into the free compounds. For example, salts of compounds of formula II can be converted by treatment with a base, such as an alkali metal hydroxide, a metal carbonate or hydrogen carbonate, or ammonia, or with another salt-forming base mentioned initially, or with an acid, such as a mineral acid, e.g. hydrochloric acid, or with another salt-forming acid mentioned initially.

The following examples serve to illustrate the invention; the temperatures are indicated in degrees Celsius, and pressures in mbar.

### EXAMPLE 1

### Bis-(3-isovaleroyl-6-methoxy-phenyl)-carbonate

7.2 g of isovaleroyl chloride are dissolved in 30 ml of methylene chloride and mixed with 10.6 g of water-free aluminium chloride. Then, a total of 5.5 g of guaiacol carbonate is added in portions, whereby the reaction temperature is maintained at 20°, if necessary by cooling. The mixture is stirred for 1 hour at room temperature, poured onto ice, extracted with ethyl acetate, the organic phase separated, dried over sodium sulfate, concentrated by evaporation and crystallized from toluene. 86% of theory of the title compound is obtained, with a m.p. of 120-122°.

### EXAMPLE 2

### 1-(3-hydroxy-4-methoxy-phenyl)-3-methyl-butan-1-one

A solution of 7.0 g of bis-(3-isovaleroyl-6-methoxyphenyl)carbonate in 20 ml of methanol is mixed with 7 ml of 30% sodium hydroxide solution and heated at reflux whilst stirring for 1½ hours. The mixture is cooled to room temperature, mixed with 50 ml of water, slightly acidified with acetic acid, extracted with toluene and evaporated to dryness. The residue, which is oily at first, crystallizes upon standing. 97% of theory of the title compound is obtained, with a m.p. of 51-53°.

### EXAMPLE 3

### 1-[3-(3-hydroxypropyloxy)-4-methoxy-phenyl]-3-methyl-butan-1-one

55 g of potassium carbonate and 30 ml of 3-chloro-1-propanol are added to a solution of 55 g of 1-(3-hydroxy-4-methoxy-phenyl)-3-methyl-butan-1-one in 250 ml of acetonitrile, and stirred at reflux for 20 hours. The suspension is filtered and concentrated by evaporation. 81 g of the title compound are obtained as the residue of evaporation. It crystallizes upon cooling, m.p. 38-40° C.

### EXAMPLE 4

### 1-[4-methoxy 3-(3-methoxypyropyloxy)-phenyl]-3-methyl-butan-1-one

40 g of powdered potassium hydroxide and 30 ml of dimethyl sulfate are added at 22-26° C. to a solution of 50 g of 1-[3-(3-hydroxypropyloxy)-4-methoxy-phenyl]-3-methyl-butan-1-one in 150 ml of toluene. The mixture is stirred for 8 hours at 40° C. Then, 150 ml of water are added, and stirring continues for a further 12 hours at room temperature. The organic solvent is separated and concentrated, and the residue distilled under a high vacuum (220° C., 0.05 torr) 41 g is obtained as an oil. The total yield of examples 3 and 4 is 90% of theory.

### EXAMPLE 5

### trans-1-bromo-6-[4-methoxy-3-(3-methoxypropyloxy)-phenyl]-5-(S)-isopropyl-hex-2-en-6-one

A solution of 15.8 g of 1-[4-methoxy-3-(3-methoxypropyloxy)-phenyl]-3-methyl-butan-1-one and 9.3 g of (S)-(−)-2-amino-1-methoxy-3-phenylpropane in 100 ml of toluene is boiled whilst removing water until ca. 1 ml of water has been removed. Ca. 60 ml of toluene is distilled off. To the remaining solution of the imine of 1-[4-methoxy-3-(3-methoxypropyloxy)-phenyl]-3-methyl-butan-1-one are added at 0° C. first of all 63 ml of a 1-molar solution of bis-trimethylsilyl-lithium amide in dry tetrahydrofuran and then 15.2 g of dimethylpropylene urea. The solution obtained is added dropwise at 0° C. to a solution of 16 g of trans 1,4-dibromobut-2-ene in 26 ml of toluene. After stirring, the mixture is acidified with diluted hydrochloric acid, separated, and the organic solution concentrated. The crude product is purified by chromatography on a column of silica gel (eluant:hexane/ethyl acetate 85:15).

### EXAMPLE 6

### trans-1-bromo-6-[4-(S)-benzyl-2-oxo-oxazolidin-3-yl]-(S)-isopropyl-hex-2-en-6-one

To a solution of 5.6 g of N-isovaleroyl-(S)-4-benzyl-oxazolidin-2-one in 12 ml of toluene are added, at 0° C., first of all 23.5 ml of a 1-molar solution of bis-trimethylsilyl-lithium amide in tetrahydrofuran, then 5.7 g of dimethylpropylene urea. The solution obtained is added dropwise at 0° C. to a solution of 6 g of trans-1,4-dibromobut-2-ene in 10 ml of toluene. After stirring, the mixture is acidified with diluted hydrochloric acid, separated, and the organic solution concentrated. The crude product is purified by chromatography on a column of silica gel (eluant:hexane/ethyl acetate 85:15).

### EXAMPLE 7

### trans-1-[4-(S)-benzyl-2-oxo-oxazolidin-3-yl]-8-[4-methoxy-3-(3-methoxypropyloxy)-phenyl]-2(S)-7(S)-diisopronyl-oct-4-en-1,8-dione

A solution of 5.6 g of 1-[4-methoxy-3-(3-methoxypropyloxy)-phenyl]-3-methyl-butan-1-one and 3.3 g of (S)-(−)-2-amino-1-methoxy-3-phenylpropane in 30 ml of toluene is boiled whilst removing water until ca. 0.3 ml of water has been removed. Ca. 20 ml of toluene is distilled off. To the remaining solution of the imine of 1-[4-methoxy-3-(3-methoxypropyloxy)-phenyl]-3-methyl-butan-1-one are added at 0° C. first of all 23.5 ml of a 1-molar solution of bis-trimethylsilyl-lithium amide in tetrahydrofuran and then 5.7 g of dimethylpropylene urea. The solution obtained is added dropwise at 0° C. to a solution of 8 g of trans-1-bromo-6-[4-(S)-benzyl-2-oxo-oxazolidin-3-yl]-5-(S)-isopropyl-hex-2-en-6-one in 10 ml of tolulene. After stirring, the mixture is acidified with diluted hydrochloric acid, separated, and the organic solution concentrated. The crude product is purified by chromatography on a column of silica gel (eluant:hexane/ethyl acetate 85:15).

### EXAMPLE 8

### trans-1-[4-(S)-benzyl-2-oxo-oxazolidin-3-yl]-8-[4-methoxy-3-(3-methoxypropyloxyl-phenyl]-2(S)-7(S)-diisopropyl-oct-4-en-1,8-dione

To a solution of 5.6 g of N-isovaleroyl-(S)-4-benzyl-oxazolidin-2-one in 12 ml of toluene are added, at 0° C., first of all 23.5 ml of a 1-molar solution of bis-trimethylsilyl-lithium amide in tetrahydrofuran, then 5.7 g of dimethylpropylene urea. The solution obtained is added dropwise at 0° C. to a solution of 8 g of trans-1-bromo-6-[4-methoxy-3-(3-methoxypropyloxy)-phenyl]-5-(S)-isopropyl-hex-2-en-6-one in 10 ml of tolulene. After stirring, the mixture is acidified with diluted hydrochloric acid, separated, and the organic solution concentrated. The crude product is purified by chromatography on a column of silica gel (eluant:hexane/ethyl acetate 85:15).

### EXAMPLE 9

### 3(S)-isopropyl-5(S)-{1(R)-bromo-3(S)-isopropyl-4-[4-methoxy-3-(3-methoxypropoxy)phenyl]-4-oxo-butyl}-tetrahydrofuran-2-one

3.6 g of N-bromosuccinimide are added at room temperature to a solution of 11 g of trans-1-[4-(S)-benzyl-2-oxo-oxazolidin-3-yl]-8-[4-methoxy-3-(3-methoxypropyloxy)-phenyl]-2(S)-7(S)-diisopropyl-oct-4-en-1,8-dione in 50 ml of methylene chloride and 50 ml of water. The mixture is stirred for 2 hours, then the organic phase is separated and concentrated. The crude product is purified by chromatography on a column of silica gel (eluant:hexane/ethyl acetate 85:15).

### EXAMPLE 10

### 3(S)-Isopropyl-5(S)-{1(S)-azido-3(S)-isopropyl-4-[4-methoxy-3-(3-methoxypropyloxyl-phenyl]-4-oxo-butyl}-tetrahydrofuran-2-one

A well stirred mixture of 25 g of 3(S)-isopropyl-5(S)-{1(R)-bromo-3(S)-isopropyl-4-[4-methoxy-3-(3-methoxypropyloxy)-phenyl]-4-oxo-butyl}-tetrahydrofuran-2-one, 12.5 g of sodium azide, 260 ml of toluene, 1.1 g of N-methyl-N,N,N-tricapryl-ammonium chloride and 38 ml of water is held at 75° C. for 40 hours. Then, the mixture is cooled to 20° C., the organic solution separated, washed with an aqueous solution of sodium nitrate and acetic acid, and concentrated.

### EXAMPLE 11

### 3(S)-Isopropyl-5(S)-{1(S)-amino-3(S)-isopropyl-4-[4-methoxy-3-(3-methoxypropyloxy)-phenyl]-butyl}-tetrahydrofuran-2-one

A solution of 11 g of 3(S)-isopropyl-5(S)-{1(S)-azido-3(S)-isopropyl-4-[4-methoxy-3-(3-methoxypropyloxy)-phenyl]-4-oxo-butyl}-tetrahydrofuran-2-one and 1.2 ml ethanolamin in 670 ml of ethanol is hydrogenated for 24 hours at room temperature and at normal pressure in the presence of 11 g of palladium on activated carbon (10%). After filtering the catalyst, the mixture is concentrated and the residue partitioned between aqueous sodium hydrogen carbonate solution and toluene. After concentrating the toluene, the product is obtained as a colourless resin.

What is claimed is:

1. Compounds of formula I 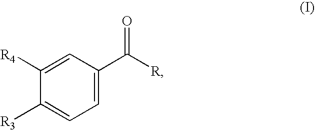
wherein
R illustrates a group of formulae Ia, Ib, Ic or Id 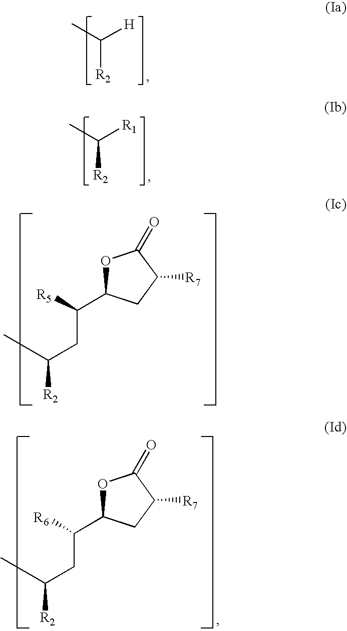
R₁ is 4-halogenbut-2-enyl,
R₂ signifies lower alkyl or cycloalkyl,
R₃ is lower alkoxy,
R₄ illustrates lower alkoxy lower alkoxy, or where R is a group of formula (Ia), it is hydroxy, hydroxy lower alkoxy or a group of formula Ie 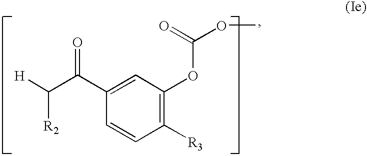
R₅ signifies reactive, esterified hydroxy,
R₆ is azido and
R₇ illustrates lower alkyl, lower alkenyl, cycloalkyl or aryl lower alkyl,
and their salts.

2. Compounds according to claim 1, wherein
R illustrates a group of formulae Ia, Ib, Ic or Id,
R₁ is 4-halogenbut-2-enyl,
R₂ is lower alkyl or 3- to 8-membered cycloalkyl,
R₃ is lower alkoxy,
R₄ illustrates lower alkoxy lower alkoxy, or where R is a group of formula (Ia), it is hydroxy, hydroxy lower alkoxy or a group of formula Ie,
R₅ is halogen or sulfonyloxy, such as lower alkane sulfonyloxy, halogen lower alkane sulfonyl, lower alkane sulfonyloxy; or benzene sulfonyloxy or naphthalene sulfonyloxy which is unsubstituted or substituted by lower alkyl, halogen and/or nitro.
R₆ is azido and
R₇ is lower alkyl, lower alkenyl, 3- to 8-membered cycloalkyl, or phenyl lower alkyl which is unsubstituted or substituted in the phenyl moiety by lower alkyl, lower alkoxy, halogen and/or nitro,
and their salts.

3. Compounds according to claim 1, wherein
R illustrates a group of formulae Ia, Ib, Ic or Id,
R₁ is 4-halogenbut-2-enyl,
R₂ is branched C₃-C₇alkyl, such as propyl,
R₃ is C₁-C₄-alkoxy, such as methoxy,
R₄ is C₁-C₄-alkoxy-C₂-C₄-alkoxy, such as 3-methoxypropyloxy, or where R is a group of formula (Ia), it is hydroxy, hydroxy-C₂-C₄-alkoxy, such as 3-hydroxypropyloxy, or a group of formula Ie,
R₅ is halogen of atomic numbers up to and including 35, such as bromine,
R₆ is azido or amino and
R₇ is branched C₃-C₇alkyl, such as propyl,
and their salts.

4. A compound according to claim 1, selected from
Bis-(3-isovaleroyl-6-methoxy-phenyl )-carbonate;
1-(3-hydroxy-4-methoxy-phenyl)-3-methyl-butan-1-one;
1-[3-(3-hydroxypropyloxy)-4-methoxy-phenyl]-3-methyl-butan-1-one
1-[4-methoxy 3-(3-methoxypropyloxy)-phenyl]-3-methyl-butan-1-one;
trans-1-bromo-6-[4-methoxy-3-(3-methoxypropyloxy)-phenyl]-5-(S)-isopropyl-hex-2-en-6-one;
3(S)-isopropyl-5(S)-{1(R)-bromo-3(S)-isopropyl-4-[4-methoxy-3-(3-methoxypropoxy)phenyl]-4-oxo-butyl}-tetrahydrofuran-2-one and
3(S)-isopropyl-5(S)-{1(S)-azido-3(S)-isopropyl-4-[4-methoxy-3-(3-methoxypropyloxy)phenyl]-4-oxo-butyl}-tetrahydrofuran-2-one.

5. Method of producing compounds of formula I 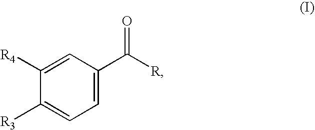
wherein
R illustrates a group of formulae Ia, Ib, Ic or Id 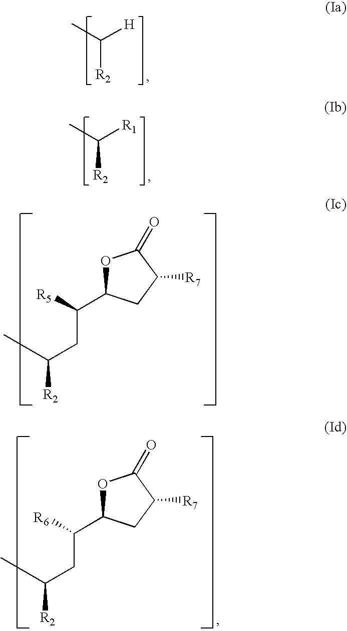
R₁ is 4-halogenbut-2-enyl,
R₂ signifies lower alkyl or cycloalkyl,
R₃ is lower alkoxy,
R₄ illustrates lower alkoxy lower alkoxy, or where R is a group of formula (Ia), it is hydroxy, hydroxy lower alkoxy or a group of formula Ie 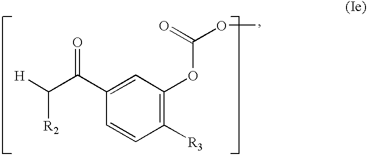
R₅ signifies reactive, esterified hydroxy,
R₆ is azido and
R₇ illustrates lower alkyl, lower alkenyl, cycloalkyl or aryl lower alkyl, and their salts, characterized in that
a) in order to produce compounds of formula I, wherein R is a group of formula Ia, R₂ is lower alkyl or cycloalkyl, R₃ is lower alkoxy and R₄ is a group of formula Ie,
a compound of formula IV 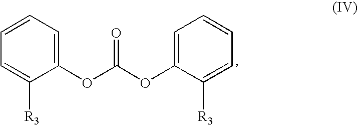
wherein R₃ has the above significance, is condensed with a compound of formula V 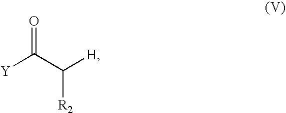
wherein R₂ has the above significance and Y is a reactive esterified hydroxy group,
b) in order to produce a compound of formula I, wherein R is a group of formula Ia, R₂ is lower alkyl or cycloalkyl, R₃ is lower alkoxy and R₄ is hydroxy, a compound of formula VI, 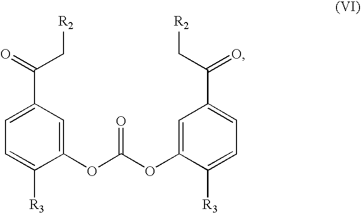
wherein R₂ and R₃ have the above significances, undergoes solvolysis to form the corresponding compounds of formula I, wherein R₄ is hydroxy,
c) in order to produce a compound of formula I, wherein R is a group of formula Ia, R₂ is lower alkyl or cycloalkyl, R₃ is lower alkoxy and R₄ is hydroxy lower alkoxy R′₄, in a compound of formula VII, 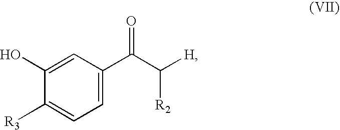
wherein R₂ and R₃ have the above significances, the hydroxy group is converted into hydroxy lower alkoxy R′₄,
d) in order to produce a compound of formula I, wherein R is a group of formula Ia, R₂ is lower alkyl or cycloalkyl, R₃ is lower alkoxy and R₄ is lower alkoxy lower alkoxy R″₄, in a compound of formula VIII, 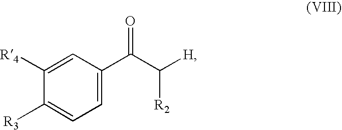
wherein R₂ and R₃ have the above significances and R′₄ is hydroxy lower alkoxy, the hydroxy lower alkoxy group is converted into lower alkoxy lower alkoxy R″₄,
e) in order to produce compounds of formula I, wherein R is a group of formula Ib, R₁ is 4-halogen-but-2-enyl, R₂ is lower alkyl or cycloalkyl, R₃ is lower alkoxy and R₄ is lower alkoxy lower alkoxy R″₄, a compound of formula IX 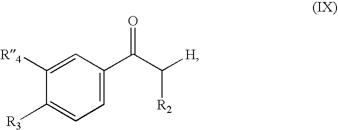
wherein R₂, R₃ and R″₄ have the above significances, is reacted first of all with a chiral primary amine which is customary for the purpose of stereoselective synthesis, and then with a 1,4-dihalogen-but-2-ene,
f) in order to produce compounds of formula I, wherein R is a group of formula Ic, R₂ is lower alkyl or cycloalkyl, R₃ is lower alkoxy, R₄ is lower alkoxy lower alkoxy R″₄, R₅ is reactive esterified hydroxy and R₇ is lower alkyl, lower alkenyl, cycloalkyl or aryl lower alkyl, a compound of formula X, 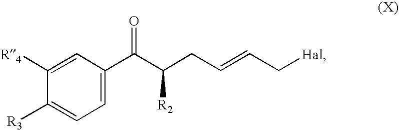
wherein R₂, R₃ and R″₄ have the above significances, and Hal is halogen, is reacted with a compound of formula XI 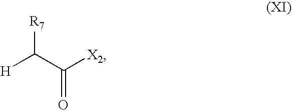
in which R₇ has the indicated significance and X₂ is a chiral amino, amido or urethane group which is customary for the purpose of stereoselective synthesis, or is a chiral alcohol group; or a 1,4-dihalogen-but-2-ene is reacted firstly with a compound of formula XI and then with the reaction product from the reaction of a compound of formula IX 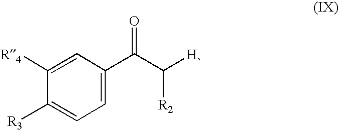
with a chiral primary amine of formula XII, which is customary for the purpose of stereoselective synthesis, 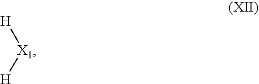
and the reaction product of formula XIII 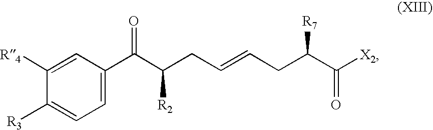
is condensed intramolecularly with lactonisation to form the corresponding compounds of formula I, wherein R is a group of formula Ic and R₅ is halogen, and if desired, halogen R₅ is converted stereoselectively into another reactive etherified hydroxy group R₅, and/or
g) in order to produce compounds of formula I, wherein R is a group of formula Id, R₂ is lower alkyl or cycloalkyl, R₃ is lower alkoxy, R₄ is lower alkoxy lower alkoxy R″₄, R₆ is azido and R₇ is lower alkyl, lower alkenyl, cycloalkyl or aryl lower alkyl, in a compound of formula XIV 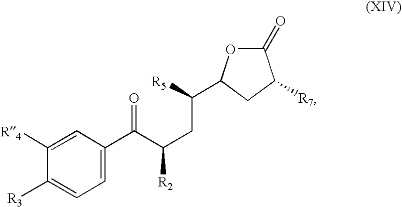
wherein R₂, R₃, R″₄ and R₇ have the above significances, and R₅ is reactive esterified hydroxy, reactive esterified hydroxy R₅ is replaced stereospecifically by azido, and if desired, a free compound obtainable according to the method is converted into a salt, or a salt obtainable according to the method is converted into the free compound or into another salt.

6. Method of producing compounds of formula II 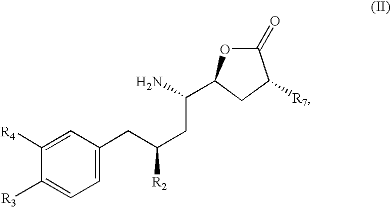
wherein
R₂ signifies lower alkyl or cycloalkyl,
R₃ is lower alkoxy,
R₄ is lower alkoxy lower alkoxy and
R₇ illustrates lower alkyl, lower alkenyl, cycloalkyl or aryl lower alkyl,
characterized in that
a) a compound of formula IV, 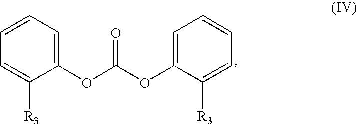
wherein R₃ has the above significance, is condensed with a compound of formula V 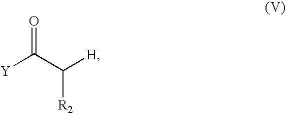
wherein R₂ has the above significance and Y is a reactive esterified hydroxy group,
b) in the obtained compound of formula VI 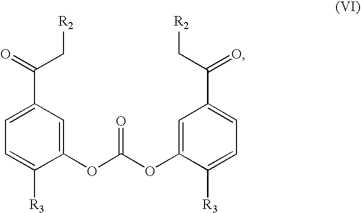
wherein R₂ and R₃ have the above significances, undergoes solvolysis to form the corresponding compounds of formula I, wherein R₄ is hydroxy,
c) in the obtained compounds of formula VII 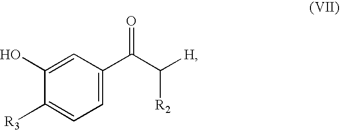
wherein R₂ and R₃ have the above significances, the hydroxy group is converted into hydroxy lower alkoxy R′₄,
d) in the obtained compounds of formula VIII 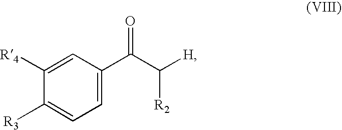
wherein R₂ and R₃ have the above significances and R′₄ is hydroxy lower alkoxy, the hydroxy lower alkoxy group is converted into lower alkoxy lower alkoxy R″₄,
e) the obtained compound of formula IX 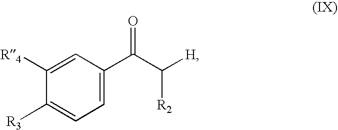
wherein R₂, R₃ and R″₄ have the above significances, is reacted first of all with a chiral primary amine which is customary for the purpose of stereoselective synthesis, and then with a 1,4-dihalogen-but-2-ene,
f) the obtained compound of formula X 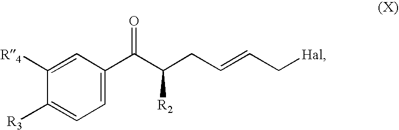
wherein R₂, R₃ and R″₄ have the above significances, and Hal is halogen, is reacted with a compound of formula XI 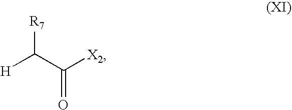
in which R₇ has the indicated significance and X₂ is a chiral amino, amido or urethane group which is customary for the purpose of stereoselective synthesis, or is a chiral alcohol group; or a 1,4-dihalogen-but-2-ene is reacted firstly with a compound of formula XI and then with the reaction product from the reaction of a compound of formula IX 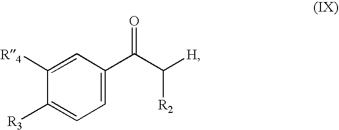
with a chiral primary amine of formula XII, which is customary for the purpose of stereoselective synthesis, 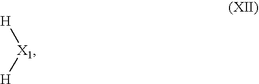
and the reaction product of formula XIII 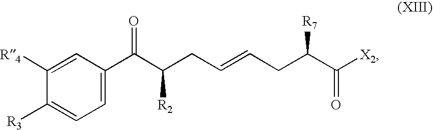
is condensed intramolecularly with lactonisation to form the corresponding compounds of formula I, wherein R is a group of formula Ic and R₅ is halogen, and if desired, halogen is converted stereoselectively into another reactive etherified hydroxy group R₅,
g) in the obtained compounds of formula XIV 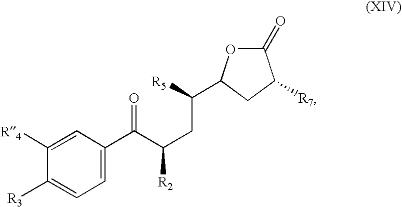
wherein R₂, R₃, R″₄ and R₇ have the above significances, and R₅ is reactive esterified hydroxy, reactive esterified hydroxy R₅ is replaced stereospecifically by azido, and
h) in a compound of formula I, wherein R is a group of formula Id, R₂ is lower alkyl or cycloalkyl, R₃ is lower alkoxy, R₄ is lower alkoxy lower alkoxy, R₆ is azido and R₇ is lower alkyl, lower alkenyl, cycloalkyl or aryl lower alkyl, the azido group R₆ is reduced to amino, and at the same time or subsequently, the benzoyl group is reduced to the corresponding benzyl group, and if desired, a free compound obtainable according to the method is converted into a salt, or a salt obtainable according to the method is converted into the free compound or into another salt.
